(19) United States
(12) Patent Application Publication
(10) Pub. No.: US 2006/0148020 A1
(43) Pub. Date: Jul. 6, 2006

(54) Inhibitors of gst a3-3 and gst a1-1 for the treatment of cancer

(76) Inventor: Bengt Mannervik, Bromma (SE)

Correspondence Address: FISH & RICHARDSON PC, P.O. BOX 1022, MINNEAPOLIS, MN, 55440-1022, US

(21) Appl. No.: 10/534,998

(22) PCT Filed: Nov. 24, 2003
(86) PCT No.: PCT/SE03/01817

§ 371 Date: Nov. 9, 2005

(30) Foreign Application Priority Data
Nov. 22, 2002 (SE) 02034791

(51) Int. Cl. C12Q 1/48; A61K 31/28

(52) U.S. Cl. 435/15; 514/492

(57) ABSTRACT

The present invention relates to a novel drug target, glutathione transferase (GST), preferably GST A3-3, as target for treatment of cancer and other diseases responsive to inhibition of steroid hormone production. The present invention also relates to a method for screening of compounds or drug candidates that modulate, preferably inhibit, the activity of GST, in which method GST is used as a drug target. The invention further relates to the use of inhibitors of GST A3-3 for production of a drug for treatment of steroid hormone dependent diseases, such as for treatment of cancer, preferably prostate cancer or breast cancer. The present invention also relates to a method for treating cancer or steroid hormone dependent diseases, comprising administering, e.g., a non-steroidal compound that modulates the tissue concentration of GST A3-3 or inhibits the enzymatic activity of GST A3-3, to a human in need of such a treatment.

### FIELD OF THE INVENTION

The present invention relates to a novel drug target. More precisely, glutathione transferase (GST) as target for treatment of cancer and other diseases responsive to inhibition of steroid hormone production. Preferably, the GST is GST A3-3 with steroid isomerase activity.

### BACKGROUND OF THE INVENTION

Prostate cancer and breast cancer are two major forms of malignant disease, which affect a significant proportion of the population. Tumor growth in both cases is often dependent on steroid hormones and an important therapeutic approach involves ablation of hormone production and blockage of the hormone receptor.

Steroid hormone biosynthesis proceeds from cholesterol to androgens (e.g. testosterone and dihydrotestosterone) and estrogens (e.g. progesterone and estradiol) via a series of metabolic intermediates. An obligatory step in each pathway leading to the respective hormones involves the isomerization of the Δ⁵-double bond to the Δ⁴-double-bond in the steroid structure. The isomerization is preceded by oxidation of the 3β-hydroxy compound into a 3-keto steroid, catalyzed by 3β-hydroxysteroid dehydrogenase. This dehydrogenase has been shown to have an associated steroid isomerase activity.

Glutathione transferases, GSTs, occur in multiple forms (1) and are present in all cellular fractions. The mammalian GSTs can be divided into soluble and membrane-bound enzymes. They are traditionally regarded as detoxication enzymes constituting the main cellular defense against electrophilic compounds that cause mutations, cancer and other degenerative diseases. However, the number of homologous GST genes in eukaryotic cells, including human, has been estimated to exceed 30, and it is becoming clear that some GSTs have other specific roles in relation to physiologically relevant substrates. Therefore, it is misleading to consider GSTs as limited to general detoxication of electrophiles, since some GSTs have roles in the metabolism of well-defined cellular substrates. The recently discovered GST A3-3 appears to have such a different role in double-bond isomerizations of steroids in hormone biosynthesis and should properly be regarded as a steroid isomerase rather than a detoxication enzyme (2).

The enzyme is present in steroidogenic organs such as testis, ovary, placenta and the adrenal gland, but not in significant amounts in other tissues such as liver, thymus, skeletal muscle and brain (2). A putative GST in the human adrenal cell line H295R is markedly induced by adrenocorticotropic hormone (ACTH), a pituitary peptide that stimulates steroid hormone synthesis (3).

It is known that GSTs functioning as cellular detoxication enzymes are inhibited by a wide variety of agents in vitro (1). The different GSTs differ widely in their sensitivities to the inhibitors, whereby a given GST may be strongly inhibited by a compound that has no effect on another GST. Some GST inhibitors have been shown to be effective in cellular systems and in clinical trials. However, inhibition data have not previously been obtained for the recently discovered GST A3-3/steroid isomerase (2) and known inhibitors may be ineffective in the steroid isomerase reaction.

### SUMMARY OF THE INVENTION

According to the present invention glutathione transferase (GST), preferably GST A3-3/steroid isomerase, is provided as a new target for chemotherapy, based on its contribution to double-bond isomerizations in steroid biosynthesis. GST A3-3 has selective tissue distribution and shows high catalytic activity in the isomerization of both Δ⁵-androstene-3,17-dione and Δ⁵-pregnene-3,20-dione (FIG. 1). The present inventor has shown that the catalytic efficiency of GST A3-3 is 200-fold higher than the steroid isomerase activity of 3β-hydroxysteroid dehydrogenase. The invention is primarily concerned with cancer in the prostate, but the principle of inhibiting steroid hormone production is also applicable to steroid-responsive cancer in the breast and in other organs. Further, it is applicable to other steroid hormone-dependent diseases such as Cushing's syndrome.

Thus, in a first aspect the invention relates to the use of glutathione transferase (GST) as a drug target for screening of compounds that inhibit the activity of GST for treatment of steroid hormone dependent diseases, such as for treatment of cancer, preferably prostate cancer and breast cancer. Inhibition of activity is also meant to include reduction of the tissue level of catalytically active GST protein by inhibiting its biosynthesis or promoting its degradation.

The GST is preferably GST A3-3. Preferably, pharmaceutically acceptable compounds, which inhibit the activity of GST A3-3 or GST A1-1, are screened for. Thus, the present invention relates to a method for screening of compounds or drug candidates that modulate, preferably inhibit, GST in which method GST is used as a drug target. Such a screening assay may for example be performed as in high throughput screening.

In a second aspect, the invention relates to the use of inhibitors of GST A3-3 or GST A1-1 identifiable by said screening method as a medicament. Said medicament can be used for treatment of steroid hormone dependent diseases, such as for treatment of cancer, preferably the cancer is prostate cancer or breast cancer.

Examples of compounds to be used according to the invention include GST inhibitors having the following formula: 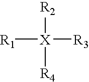
wherein R₁, R₂, R₃ and R₄ can be alkyl groups, such as methyl, ethyl, propyl, butyl, pentyl, hexyl; aryl groups, such as phenyl or substituted phenyl, preferably substituted with lower alkyl, hydroxyl or alkoxy groups; or chemical derivatives or combinations of these groups; the R₁, R₂, R₃ and R₄ groups can be linear; branched, such as substituted with lower alkyl, hydroxyl or alkoxy groups; or cyclic, such as cyclopentyl and cyclohexyl; the R₁, R₂, R₃ and R₄ groups can contain heteroatoms such as O, S, and N. The inhibitors can be stereoisomers depending on the nature and spatial orientation of the groups surrounding X; two, three or four of the R₁, R₂, R₃ and R₄ groups can be linked together and have a bidentate, tridentate or tetradentate coordination with the central atom X; Alternatively, one, two, three or four of R₁, R₂, R₃ and R₄ can be Cl, Br, I, O, S, Se, carboxylate ions such as acetate and homologs, or other chemical ligands with an electron-donating group coordinated to X.

X═Ge, Sn, Pb or similar electrophilic atoms.

The GST inhibitors preferably contain tin (Sn) as electrophilic atom, since such compounds combine moderate toxicity with strong inhibition of the target enzyme. The tin atoms of the inhibitors can have different oxidation states, such as Sn(II) or Sn(IV), and the coordination number of the ligands can be 2, 3, 4, 5 or 6.

Preferably, one of R₁-R₄ is Cl, Br or acetate and the other substituents are ethyl, butyl or phenyl.

A second group of inhibitors are steroids, steroid derivatives or steroid-mimetic compounds.

A third group of inhibitors are peptides, peptide derivatives or peptidomimetics with structural similarities to glutathione (γ-glutamyl-cysteinyl-glycine).

In a third aspect, the invention relates to a method for treating cancer or steroid hormone dependent diseases, comprising administering a compound that inhibits the enzymatic activity of GST A3-3/steroid isomerase (and/or GST A1-1) to a human in need of such a treatment. Such inhibition also includes reduction of the tissue level of active GST A3-3/steroid isomerase protein (and/or GST A1-1 protein). This reduction could be accomplished by inhibitory nucleic acid such as oligonucleotides, inhibitory RNA (siRNA or RNAi) or PNA (peptide nucleic acids) that have an effect on the gene expression and biosynthesis of the GST protein. Methods for suppression of gene expression by specific binding to the targeted gene or its corresponding RNA are well established within the field and reagents are commercially available for this purpose.

The human in need of the above-mentioned treatment may be an individual in need of treatment of steroid hormone dependent cancer or treatment of other steroid hormone dependent diseases, such as Cushing's syndrome.

In one embodiment the human is a male who suffers from prostate cancer. In another embodiment the human is a female who suffers from breast cancer.

Domestic animals (e.g. horse, dog) in need of steroid hormone suppression represent still another group of biological species to which the invention applies.

### BRIEF DESCRIPTION OF THE DRAWINGS

The invention will be described more closely below with reference to some non-limiting examples and figures.

FIG. 1. Metabolic pathways leading from cholesterol to steroid hormones such as testosterone (and further to dihydrotestosterone) and progesterone (and further to estradiol). The hormones act via binding to the androgen and estrogen receptors, respectively, and promote growth of hormone responsive prostate and breast cancer. GST A3-3 catalyzes essential steroid isomerizations in the respective pathways and the invention involves this enzyme as a target for hormone responsive disease.

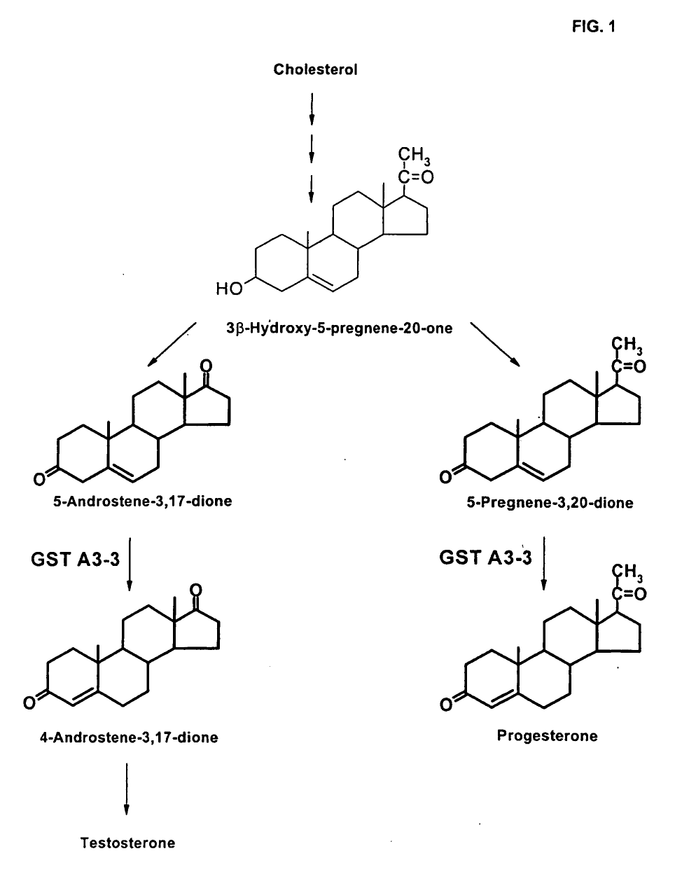

FIG. 2. Alternative reactions for measuring the inhibition of GST A3-3 in vitro. All three reactions can be monitored spectrophotometrically using purified enzyme and glutathione (GSH): (A) Δ⁵-androstene-3,17-dione; (B) 1-chloro-2,4-dinitrobenzene; and (C) phenethylisothiocyanate. Addition of an inhibitor will decrease the rate of the reaction catalyzed by GST A3-3.

### DETAILED DESCRIPTION OF THE INVENTION

Experimental Procedures

Materials—1-Chloro-2,4-dinitrobenzene (CDNB) and reduced glutathione (GSH) can be purchased from Sigma (St. Louis, Mo.), phenethylisothiocyanate from Aldrich (Milwaukee, Wis.), and Δ⁵-androstene-3,17-dione from Steraloids Inc. (Newport, R.I.).

Expression and purification of GSTs—Human GST A3-3 and its homologous GST proteins of the Alpha class were expressed from corresponding cDNA carried by the pET-21a(+) vector in E. coli BL-21(DE3) (2). The cells were grown to OD₆₀₀=0.7 and expression was induced by addition of 1 mM IPTG. The cells were grown for four hours, collected by centrifugation, and lysed using ultrasonication. The lysate was desalted on a PD-10 gel filtration column (Amersham Biosciences) and the proteins were eluted in 20 mM sodium phosphate, pH 7.0, and were subsequently loaded onto a HiTrap SP cation exchanger (Amersham Biosciences). The proteins were eluted using a salt gradient. This single purification step yielded highly pure enzymes as confirmed by SDS-PAGE stained with Coomassie Brilliant Blue.

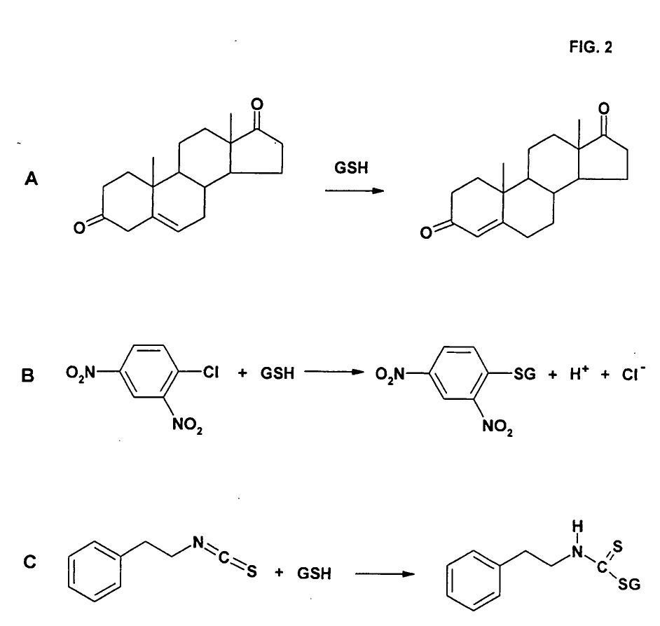

Specific activity measurements—The specific activities of GST A3-3 were determined for the isomerization reaction with Δ⁵-AD (FIG. 2A), the conjugation reaction with 1-chloro-2,4-dinitrobenzene (CDNB) and GSH (FIG. 2B), and for the addition of GSH to phenethylisothiocyanate (FIG. 2C). The reactions were monitored spectrophotometrically at 30° C. The isomerization of 100 μM Δ⁵-AD was followed at 248 nm in 25 mM sodium phosphate buffer, pH 8.0, in the presence of 1 mM GSH. The extinction coefficient for the product Δ⁴-AD is 16,300 M⁻¹cm⁻¹. Specific activity measurements were performed in 0.1 M sodium phosphate, pH 6.5, with 1 mM CDNB in the presence of 1 mM GSH as described (4), and with 0.1 mM phenethylisothiocyanate in the presence of 1 mM GSH (2).

Examples of Specific Inhibitors of Alpha Class GSTs

Enzyme activities were determined in the standard assay system and the concentration of the inhibitor giving 50% inhibition of the activity (IC₅₀) was determined.

Even if the compounds are inhibiting several GSTs, some inhibitors display high selectivity for a given GST (1). The present inventor has shown that this applies also to homologous members of the same GST class (Table 1). Selective inhibition is desirable to avoid interference with non-targeted GST-catalyzed reactions and to minimize possible toxic side effects. Without any extensive screening, inhibitors of GST A3-3 effective in the nanomolar concentration range have already been identified. These inhibitors also display selectivity among GST A3-3 and other human Alpha class members (Table 1). However, the related GST A1-1 has approximately 5% of the specific activity of GST A3-3 in the isomerization of androstenedione (2), and it may be advantageous to inhibit GST A1-1 in addition to GST A3-3. By use of multivariate cluster analysis of inhibition data it is possible to optimize discrimination among the enzymes.

TABLE 1
Differential inhibition of Alpha class glutathione transferases
demonstrated by using organometallic compounds. The IC₅₀ values
are the inhibitor concentrations giving 50% inhibition of the
GST-catalyzed reaction.

| | IC₅₀ Values (μM) | | |
|---|---|---|---|
| Inhibitor | GST A1-1 | GST A2-2 | GST A3-3 |
| Et₃GeCl | 56 | 0.8 | 67 |
| Bu₃SnAc | 0.018 | 0.41 | 0.018 |
| Et₃SnBr | 5.7 | 0.19 | 0.69 |
| Ph₃PbCl | 0.0046 | 0.084 | 0.013 |
| Ph₃SnAc | 0.16 | Nd | 0.16 |
| Et₃PbCl | 2 | Nd | 2.3 |
| Ph₃PbBr | 0.0086 | Nd | 0.16 |

Et, Bu, and Ph are ethyl, n-butyl, and phenyl, respectively;
Nd = not determined.

Other examples of compounds inhibiting GST A3-3 is a steroid such as Δ⁵-androsten-3β-ol-17-one or a structurally similar compound.

Other possible inhibitors that inhibit GST A3-3 can be found among peptides, peptide derivatives or peptidomimetic compounds having structural similarities with glutathione (i.e., γ-glutamyl-cysteinyl-glycine), and which are S-substituted, or otherwise substituted glutathione derivatives. Substituents include alkyl, aryl and aralkyl groups. Such inhibitors can for example be S-hexyl-glutathione or S-p-bromobenzyl-glutathione.

### REFERENCES

1. B. Mannervik and U. H. Danielson (1988) Glutathione transferases—structure and catalytic activity, CRC Crit. Rev. Biochem. 23, 283-337.
2. A.-S. Johansson and B. Mannervik (2001) Human glutathione transferase A3-3, a highly efficient catalyst of double-bond isomerization in the biosynthetic pathway of steroid hormones, J. Biol. Chem. 276, 33061-33065.
3. T. Stark, L. Mankowitz and J. W. DePierre (2002) Expression of glutathione transferase isoenzymes in the human H295R adrenal cell line and the effect of forskolin, J. Biochem. Mol. Toxicol. 16, 169-173.
4. B. Mannervik and P. Jemth (1999) Measurement of glutathione transferases, in “Current Protocols in Toxicology” (M. D. Maines, L. G. Costa, D. J. Reed, S. Sassa, and I. G. Sipes, eds.), pp. 6.4.1-6.4.10, John Wiley & Sons, New York.

1. Method of screening for compounds that suppress the concentration of active glutathione transferase (GST) protein or inhibit the steroid isomerase activity of glutathione transferase (GST), wherein a glutathione transferase (GST) is used as a drug target.

2. Method according to claim 1, wherein the GST used is GST A3-3.

3. Method according to claim 1, wherein the GST used is GST A1-1.

4. An inhibitor that inhibits the steroid isomerase activity of glutathione transferase (GST) identifiable by the method according to claim 1.

5. An inhibitor lowering the tissue concentration of active glutathione transferase (GST) identifiable by the method according to claim 1.

6. An inhibitor according to claim 4, wherein the inhibitor is a compound having the following formula: 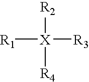
wherein R₁, R₂, R₃ and R₄ can be alkyl groups, such as methyl, ethyl, propyl, butyl, pentyl, hexyl; aryl groups, such as phenyl or substituted phenyl, preferably substituted with lower alkyl, hydroxyl or alkoxy groups; or chemical derivatives or combinations of these groups; the R₁, R₂, R₃ and R₄ groups can be linear; branched, such as substituted with lower alkyl, hydroxyl or alkoxy groups; or cyclic, such as cyclopentyl and cyclohexyl; the R₁, R₂, R₃ and R₄ groups can contain heteroatoms such as O, S, and N; alternatively, one, two, three or four of R₁, R₂, R₃ and R₄ can be Cl, Br, I, O, S, Se, carboxylate ions such as acetate and homologs, or other chemical ligands with an electron-donating group coordinated to X;
X═Ge, Sn, Pb or similar electrophilic atoms;
as well as stereoisomers of the inhibitor.

7. An inhibitor according to claim 6, wherein X is Sn.

8. An inhibitor according to claim 6, wherein one of R₁-R₄ is Cl, Br or acetate and the other substituents are ethyl, butyl or phenyl.

9. An inhibitor according to claim 4, wherein the inhibitor is a steroid, steroid derivative or steroid-mimetic compound.

10. An inhibitor according to claim 9, wherein the inhibitor is Δ⁵-androsten-3β-ol-17-one or a structurally similar compound.

11. An inhibitor according to claim 4, wherein the inhibitor is a peptide, peptide derivative or peptidomimetic compound with structural similarities to glutathione.

12. An inhibitor according to claim 11, wherein the inhibitor is an S-substituted, and/or otherwise substituted, glutathione derivative where the substituents may be alkyl, aryl and aralkyl groups.

13. An inhibitor according to claim 12, wherein the inhibitor is S-hexyl-glutathione or S-p-bromobenzyl-glutathione.

14. An inhibitor according to claim 5, wherein the inhibitor is an inhibitory nucleic acid such as an oligonucleotide, an inhibitory RNA (siRNA or RNAi) or PNA (a peptide nucleic acid).

15. An inhibitor according to claim 4 for use as a medicament.

16. A medicament according to claim 15 for use in treatment of steroid hormone dependent diseases in a mammal.

17. A medicament according to claim 16 for use in treatment of steroid hormone dependent cancer.

18. A medicament according to claim 17 for use in treatment of prostate cancer.

19. A medicament according to claim 17 for use in treatment of breast cancer.

20. A medicament according to claim 16 for use in treatment of Cushing's syndrome.

21. A method for treating cancer or steroid hormone dependent diseases, comprising administering a compound that suppresses the concentration of active glutathione transferase (GST) protein or inhibits the steroid isomerase activity of glutathione transferase (GST) of GST A3-3 and/or GST A1-1 to a human in need of such a treatment.

22. A method according to claim 21, wherein the human is a male who suffers from prostate cancer.

23. A method according to claim 21, wherein the human is a female who suffers from breast cancer.

24. A method according to claim 21, wherein the human is suffering from Cushing's syndrome.
